(19) United States
(12) Patent Application Publication
(10) Pub. No.: US 2012/0035646 A1
(43) Pub. Date: Feb. 9, 2012

(54) BISTABLE BODY LUMEN FILTER ANCHORS

(75) Inventor: Kelly J. McCrystle, Menlo Park, CA (US)

(73) Assignee: ABBOTT LABORATORIES VASCULAR ENTERPRISES LIMITED, Dublin (IE)

(21) Appl. No.: 12/851,674

(22) Filed: Aug. 6, 2010

(51) Int. Cl. A61F 2/01

(52) U.S. Cl. 606/200

(57) ABSTRACT

A body lumen filter includes a plurality of bistable anchors configured to move between a stable pre-deployed state and a stable deployed state. In the stable deployed state the stable pre-deployed diameter the bistable anchors define a pre-deployed anchor diameter and in the stable deployed diameter the bistable anchors define a deployed anchor diameter, the deployed diameter being larger than the pre-deployed diameter. A filtering structure is operatively associated with the bistable anchors.

### BACKGROUND

1. Field of the Invention

The present disclosure relates generally to medical devices and to body lumen filters in particular and methods for filtering a body lumen. More particularly, embodiments of the invention relate to body lumen filters with bistable anchors.

2. Background and Relevant Art

Surgical procedures, including both invasive as well as minimally-invasive procedures, save countless lives each year. However, the instruments and processes used during such procedures sometimes create additional challenges. For example, many minimally invasive procedures are performed using highly specialized surgical tools that are introduced to the procedure site by way of the patient's vasculature. For example, a catheter is introduced into the patient's vasculature by way of a small incision. The catheter is then advanced into proximity with the procedure site.

Thereafter, surgical tools may be advanced to the procedure site through the catheter. With the surgical tools thus at the procedure site, the surgical tools are then manipulated from the outside of the body. Accordingly, a surgical procedure can be performed with only a small incision.

While such an approach can reduce the invasiveness of performing a surgical procedure, this approach can cause additional challenges. In particular, as the catheter and/or surgical devices are advanced through the vasculature, their passage can cause arterial plaques, clots, or other debris commonly referred to as emboli to become dislodged and move with the blood as it circulates through the vasculature. As the emboli move downstream, they can encounter plaque or other obstructions within the bloodstream to form new clots or obstructions in the bloodstream. Such obstructions can result in partial or complete blockage of vessels supplying blood and oxygen to critical organs, such as the heart, lungs and brain.

### BRIEF SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

A body lumen filter includes a plurality of bistable anchors configured to move between a stable pre-deployed state and a stable deployed state. In the stable deployed state the stable pre-deployed diameter the bistable anchors define a pre-deployed anchor diameter and in the stable deployed diameter the bistable anchors define a deployed anchor diameter, the deployed diameter being larger than the pre-deployed diameter. A filtering structure is operatively associated with the bistable anchors.

These and other features of the present invention will become more fully apparent from the following description and appended claims, or can be learned by the practice of the invention as set forth hereinafter.

### BRIEF DESCRIPTION OF THE DRAWINGS

In order to describe the manner in which at least some of the advantages and features of the invention can be obtained, a more particular description of the invention briefly described above will be rendered by reference to specific embodiments thereof which are illustrated in the appended drawings. Understanding that these drawings depict only typical examples and are not therefore to be considered to be limiting of the invention's scope, examples will be described and explained with additional specificity and detail through the use of the accompanying drawings in which:

FIG. 1A illustrates a body lumen filter in a pre-deployed state according to one example;

FIG. 1B illustrates a body lumen filter in a deployed state according to one example;

FIG. 2A illustrates a body lumen filter in a pre-deployed state being introduced to a body lumen by a deployment device according to one example;

FIG. 2B illustrates a body lumen filter being moved toward a deployed state in the body lumen according to one example;

FIG. 2C illustrates the body lumen filter in a deployed state in the body lumen according to one example;

FIG. 2D illustrates a removal step of the body lumen filter according to one example;

FIG. 3A illustrates a body lumen filter in a pre-deployed state according to one example;

FIG. 3B illustrates a body lumen filter in a deployed state according to one example;

FIG. 4A illustrates a body lumen filter in a pre-deployed state according to one example;

FIG. 4B illustrates a body lumen filter in a deployed state according to one example;

FIG. 5A illustrates a body lumen filter in a pre-deployed state being introduced to a body lumen by a deployment device according to one example;

FIG. 5B illustrates first anchors of a body lumen filter being moved toward a deployed state in the body lumen according to one example;

FIG. 5C illustrates second anchors of a body lumen filter being moved toward a deployed state in the body lumen according to one example;

FIG. 5D illustrates first anchors of a body lumen filter being moved toward a pre-deployed state according to one example;

FIG. 5E illustrates second anchors of a body lumen filter being moved toward a pre-deployed state in the body lumen according to one example;

FIG. 6A illustrates a body lumen filter in a pre-deployed state according to one example;

FIG. 6B illustrates a body lumen filter in a deployed state according to one example; and

FIG. 7 illustrates exemplary deployment of a body lumen filter in a patient according to one example.

### DETAILED DESCRIPTION

Devices and systems are provided herein for filtering a body lumen. By way of example only, a body lumen may include a blood vessel. Filtering may be performed by body lumen filters. For instance, embodiments of body lumen filters (e.g. including vena cava and/or other lumen filters), are described. In some embodiments, the filter devices or body lumen filters capture emboli within the lumen at safe locations. Vena cava filters, by way of example are devices that are implanted in the inferior vena cava, providing a mechanical barrier to undesirable particulates or emboli. The filters may be used to filter peripheral venous blood clots and other particulates or emboli, which if remaining in the blood stream, can migrate in the pulmonary artery or one of its branches or other location in the body and cause harm.

Components of body lumen filters also are described. These components may include anchors and/or other components, including components configured to anchor a body lumen filter to a wall. These components include bistable anchors that move between a stable, pre-deployed state and a stable, deployed state by passing through a transition state. When the deflection of a bistable anchor is on one side of the transition state, the bistable anchor automatically moves to that side of the bistable state. Accordingly, the stable nature of the anchors in the pre-deployed state alone can help maintain the anchors in the pre-deployed state until a force is applied to move the anchors to the deployed state. In at least one example, the force applied to move the anchors between the stable states is substantially less than a force associated with plastic deformation of the anchors. A bistable anchors can aid in the placement of a body lumen filter and/or in the extraction of a body lumen filter from a deployment site. Some body lumen filters may be designed to capture and/or lyse particles of a particular size, for example, when moving or converting to a pre-deployed state after having been deployed in a deployed state.

Many body lumen filters may include hooks and/or other anchoring devices that pierce the inner wall of the body lumen to prevent filter migration. In some cases, piercing the inner wall of the body lumen may not be desirable. For instance, where the body lumen is already weakened. Body lumen filters that do not include hooks and/or other anchoring devices that pierce the inner wall of the body lumen may be subject to filter migration.

Thus, embodiments relating to a body lumen filter with anchors having relatively large surface areas and methods for filtering a body lumen may be useful for facilitating filtering of a body lumen. Further, the bistable anchors described herein can move between a stable, pre-deployed state toward a stable, deployed state by the application of generally radially oriented forces that are substantially less than forces associated with plastic deformation.

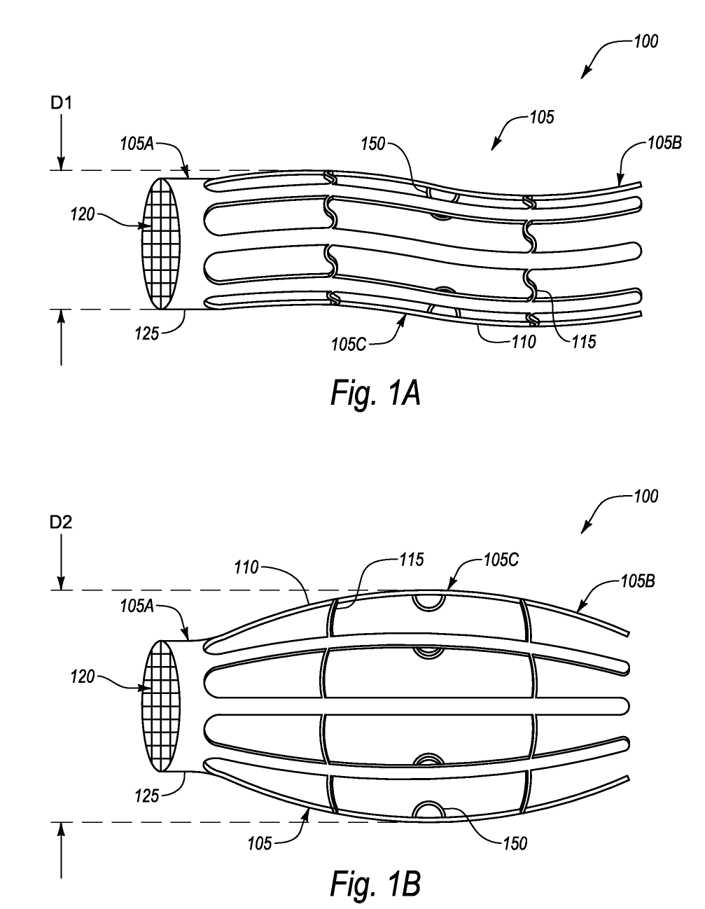

FIG. 1A illustrates a body lumen filter 100 for filtering a body lumen, such as a blood vessel. As illustrated in FIG. 1A, the body lumen filter 100 includes a body 105 having a plurality of bistable anchors 110. In at least one example, the bistable anchors 110 can anchor the body lumen filter 100 to a wall of a body lumen. Accordingly, the bistable anchors 110 can also serve as anchoring structures. The anchoring structures can keep the filter 100 in the deployment site and ensure that the filter 100 is fixed or is less susceptible to filter migration. In addition, the body lumen filter 100 can also include lateral struts 115 that are interconnected with the bistable anchors 110 in such a manner as to allow the body lumen filter 100 to be moved from a stable pre-deployed state shown in FIG. 1A through a transition state to a stable, deployed configuration shown in FIG. 1B. The lateral struts 115 shown can have any suitable configuration. In at least one example, the bistable anchors 110 and the lateral struts 115 cooperate to form a lateral filtering structure. The lateral struts 115 can also be configured to move between a stable, pre-deployed state toward a stable, deployed state and thereby aid in the expansion and/or contraction of the body lumen filter 100.

In at least one example, the pre-deployed configuration can be a relatively collapsed configuration. In such examples, the body lumen filter 100 can be moved from the pre-deployed state to the deployed state by moving the bistable anchors 110 from one bistable state toward a second bistable state. As a force deflects the bistable anchors 110 from a first stable state toward a second stable state, the bistable anchors 110 move through a tipping or intermediate state. If the force deflecting the bistable anchors from the first stable state is removed before the bistable anchors 110 are past the tipping state, the bistable anchors will return to the first stable state. However, once the bistable anchors 110 have moved past the tipping state, the bistable anchors 110 will continue to deflect toward the second stable state. As a result, the bistable anchors 110 tend to spring toward the stable states away from the tipping point. Accordingly, in the pre-deployed state, a plurality of the bistable anchors 110 can remain in the pre-deployed stable state shown in FIG. 1A until sufficient forces is applied to the bistable anchors are moved past a tipping state. Any number of factors can be manipulated to provide a bistable configuration.

For example, bistable anchors 110 can have a combination of geometry and/or material properties such that a force applied to the bistable anchor 110 in an appropriate radial direction causes the bistable anchor 110 to move from one bistable position toward the other bistable position. The force associated with moving the bistable anchor 110 between bistable positions can be significantly less than a force associated with plastic deformation of the same strut, such as an order of magnitude or greater difference between the forces.

In the illustrated example, the bistable anchor 110 can have an at least partially sinusoidal shape as shown in FIG. 1A such that the body lumen filter 100 has pre-deployed diameter D1. The body lumen filter 100 can be deployed by applying a radially outward force to at least one of the bistable anchors 110 and/or lateral struts 115. The radially outward force can be applied in any suitable manner.

The radially outward force causes the bistable anchors 110 to deflect toward a deployed state. In the deployed state illustrated in FIG. 1B, the bistable anchors 110 can have a generally curved or arcuate shape thereby causing the body lumen filter 100 to define a deployed diameter D2, which is greater than the pre-deployed diameter D1 of the body lumen filter 100 in FIG. 1A. Such a configuration allows the body lumen filter 100 to be positioned in a body lumen while in a pre-deployed state and then moved to a deployed state. As the bistable anchors 110 move toward the deployed state, the bistable anchors 110 engage a wall of the body lumen to thereby anchor the body lumen filter 100 to the body lumen wall.

The body 105 generally includes a first or distal end 105A and a proximal end 105B. In at least one example, a filtering structure 120 is operatively associated with the distal end 105A. In particular, the bistable anchors 110 as well as the filtering structure 120 can be coupled to a support 125, such as a ring-shaped support. In the illustrated example, the support 125 can be configured to maintain its shape as the bistable anchors 110 move between the bistable states shown in FIG. 1A and FIG. 1B. In such an example, in the deployed state the first end 105A is narrower than a central portion 105C and/or proximal end 105B of the body 105. As a result, in the deployed state the body lumen filter 100 can have a generally tapered shape. Such a configuration can allow the body lumen filter 100 to be deployed to filter a flow of fluid through a body lumen filter, as will now be discussed in more detail. Such a configuration can also allow the body lumen filter 100 to be extracted from the body lumen.

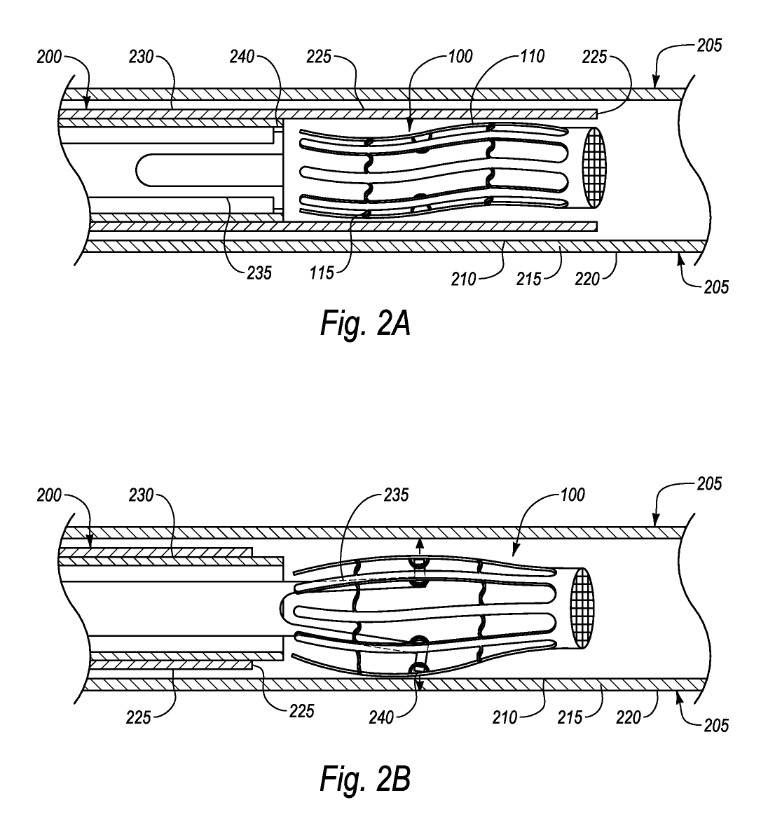

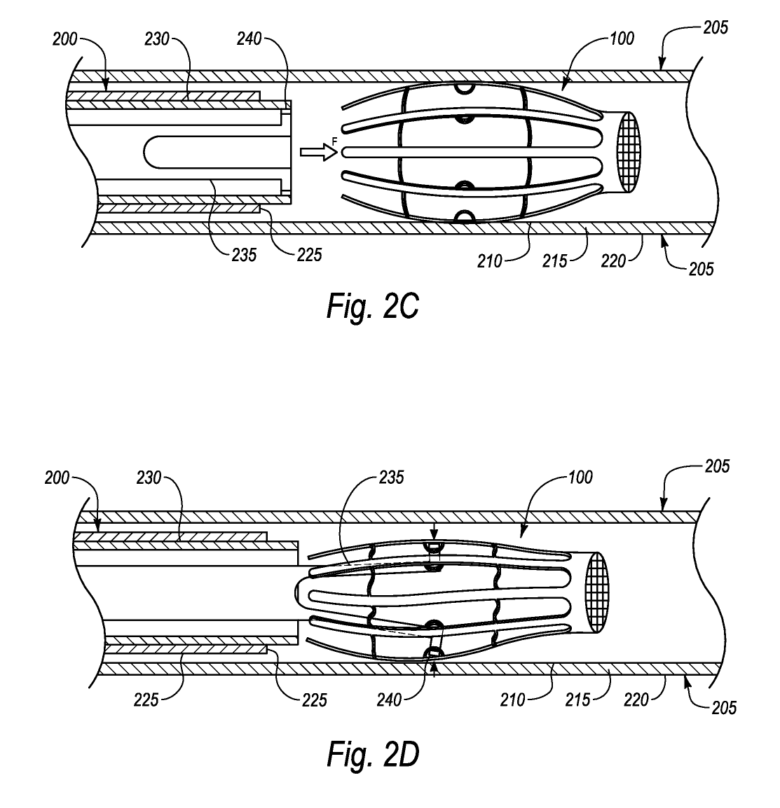

FIG. 2A illustrates the body lumen filter 100 in a pre-deployed configuration and located within a deployment device 200. The deployment device 200 is configured to deploy the filter 100 into a body lumen 205. The body lumen 205 may include an inner layer 210 (i.e. intima layer), a medial layer 215, an adventitial layer 220, or combinations thereof. The deployment device 200 can include an outer housing 225 and an inner housing 230 that may be actuated from a proximally located handle (not shown). A radial force mechanism 235 may be positioned within the inner housing 230 and may similarly be actuated from the proximally located handle. The radial force mechanism 235 can include retrieval features 240 configured to engage a corresponding engagement feature 150 (FIG. 1B) on the body lumen filter 100.

As previously discussed, the body lumen filter 100 includes bistable anchors 110 and/or lateral struts 115 that are formed in such a manner as to allow the body lumen filter 100 to be moved from the pre-deployed state illustrated in FIG. 2A to a deployed state illustrated in FIG. 2C. To deploy the body lumen filter 100, the deployment device 200 is moved near a desired location within a body lumen 205 by using a catheter or other techniques. In one example, once the deployment device 200 is near the desired location, the inner housing 230 may be advanced distally relative to the outer housing 225, thereby driving the body lumen filter 100 from the outer housing 225 toward the desired location. In another example, the deployment device 200 may be advanced to the desired location, the inner housing 230 may be advanced distally to abut the body lumen filter 100, the outer housing 225 may be retracted to deploy the body lumen filter 100, or combinations thereof.

Once the body lumen filter 100 is at the desired location within the body lumen 205, the radial force mechanism 235 can be moved from within the inner housing 230. In particular, the radial force mechanism 235 may be advanced distally relative to the inner 230 housing, the inner housing 230 may be retracted relative to the radial force mechanism 235, or combinations thereof. In at least one example, the radial force mechanism 235 can be formed of a resilient material shaped such that as the radial force mechanism 235 passes from the inner housing 230, the radial force mechanism 235 expands as illustrated in FIG. 2B.

As the radial force mechanism 235 expands it comes into contact with one or more of the bistable anchors 110. Continued expansion of the radial force mechanism 235 as it expands can exert sufficient force on the bistable anchors 110 to cause the bistable anchors 110 to deflect past the tipping state described above. Once the radial force mechanism 235 deflects the bistable anchors 110 past the tipping state, the bistable anchors 110 continue to deflect toward the deployed state, illustrated in FIG. 2C.

A body lumen filter 100 having bistable states is illustrated. Examples of the body lumen filter 100 including the bistable anchors 110 and/or lateral struts 115 can include a material made from any of a variety of known suitable materials, including metallic materials, plastic materials, composite materials, and combinations thereof. Further, the body lumen filter 100 can be formed by any number of processes, such as cutting processes, molding processes, joining processes, other processes and combinations thereof.

Continuing with the example illustrated in FIG. 2C, as the body lumen filter 100 is moved toward the deployed state, the bistable anchors 110 can be displaced to provide communication with the filter structure 120. The filter structure 120 can include openings sized to prevent particulates, such as at least one embolus, from passing through the body lumen filter 100 and/or to lyse particulates of greater than a desired size. While an embolus is trapped against body lumen filter 100, blood may continue to flow over the embolus. The flow of blood over the embolus can dissolve the embolus through the body's lysing process. Additionally, the body lumen filter 100 may be coated with a beneficial agent that may facilitate lysing of the embolus.

A blood flow F exerts a fluid force on the body lumen filter 100 that would tend to move the body lumen filter 100 in the direction of the blood flow F. The anchors 110 may resist this force to generally maintain the body lumen filter 100 in or near an intended deployment location. In particular, frictional, compressive, and/or other forces between the body lumen filter 100 and the body lumen 205 may generally maintain the body lumen filter 100 at or near the intended deployment location, as will now be described in more detail below.

As the body lumen filter 100 is moved toward the deployed state, a plurality of the bistable anchors 110 may be moved into contact with the intima layer 210 of the body lumen 205. In the deployed state, bistable anchors 110 can be separated by a distance that is slightly larger than the diameter of the body lumen 205 before the body lumen filter 100 is deployed. As a result, a tensile force can urge or press a center portion of one or more of the bistable anchors 110 into contact with the intima layer 210.

As the bistable anchors 110 are urged into contact with the intima layer 210, the intima layer 210 can deform slightly to begin to conform to the shape of the bistable anchors 110, which can result in compressive forces between the bistable anchors 110 and the body lumen 205.

Further, this deformation can increase contact between the bistable anchors 110 and the intima layer 210. Frictional forces between two objects that are in contact typically depend on the normally applied force and the coefficient of friction between the two objects. The normally applied force depends on the area of contact and the pressure applied to that area. The coefficient of friction as well as the normal force necessary to maintain the body lumen filter 100 positioned in body lumen 205 may be relatively constant. Accordingly, increasing the surface area over which the bistable anchors 110 apply the normal force can reduce the pressure the bistable anchors 110 apply to the intima layer 210 of the body lumen 205. Decreasing the pressure applied to the body lumen 205, in turn, can reduce the possibility that the bistable anchors 110 will pierce the intima layer 210.

Accordingly, the relatively large surface area of the bistable anchors 110 can help maintain the body lumen filter 100 at or near a desired deployment location in the body lumen 205. Further, the relatively large surface area of the bistable anchors 110 can reduce the likelihood that the bistable anchors 110 will penetrate through the intima layer 210 and into the medial layer 215 and/or the adventitial layer 220. Reducing penetration into the medial layer 215 may in turn reduce endothelial growth while and/or after the body lumen filter 100 is deployed.

At some point, it may be desirable to retrieve the body lumen filter 100. FIG. 2D illustrates a step for retrieving the body lumen filter 100. As illustrated in FIG. 2D, retrieving the body lumen filter 100 can include positioning the deployment device 200 such that the inner housing 230 is positioned in proximity to the body lumen filter 100 and further positioning the radial force mechanism 235 distally of the inner housing 230. Thereafter, the radial force mechanism 235 can be moved into engagement with the engagement feature 150 on the body lumen filter 100. In particular, the radial force mechanism 235 can be advanced distally beyond the inner housing 230 to allow the radial force mechanism 235 to expand.

The retrieval features 240 associated with the radial force mechanism 235 can then be moved into engagement with engagement features 150 or some other portion of the body lumen filter 100. Thereafter, the radial force mechanism 235 can be moved proximally relative to the inner housing 230 to thereby cause the retrieval features 240 to move radially inward. Engagement between the engagement features 150 and the retrieval feature 240 also causes the bistable anchors 110 to deflect radially inward toward the pre-deployed stable state. Once the bistable anchors 110 are moved past the tipping state, the bistable anchors 110 continue to deflect to the pre-deployed stable state. In the pre-deployed stable state, the body lumen filter 100 can be drawn into the outer housing 225 by drawing the radial force mechanism 225 within the outer housing 225. Once the body lumen filter 200 is located within the deployment device 200, the deployment device 200 can be removed to thereby complete retrieval of the body lumen filter 100. In the example illustrated in FIGS. 1A-2D, the filtering structure 120 and the bistable anchors 110 are coupled to a non-expanding support 125. It will be appreciated that other examples include bistable anchors in other configurations.

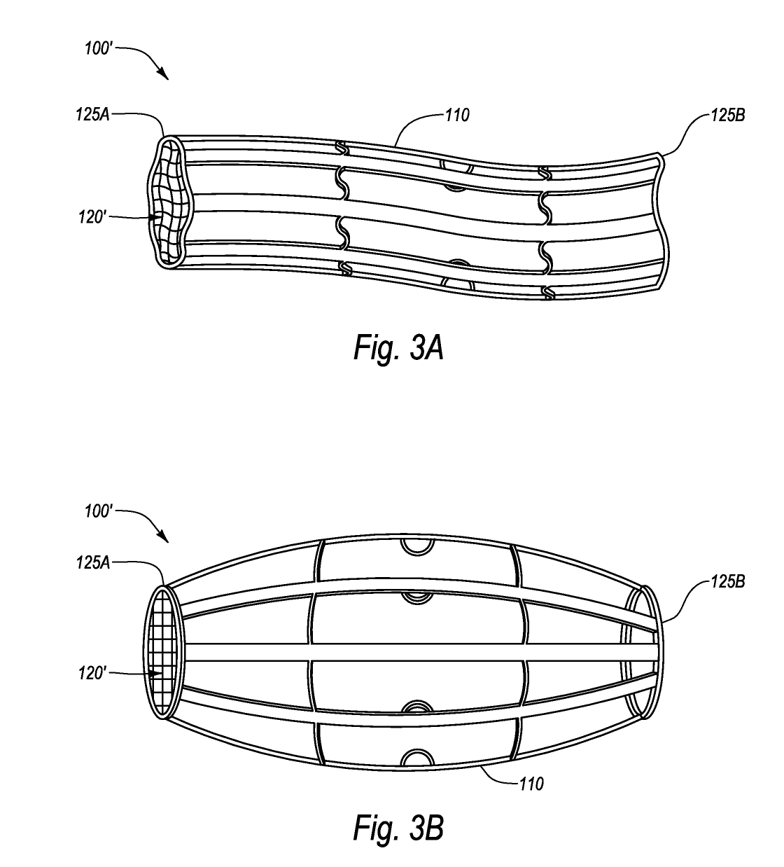

For example, FIGS. 3A and 3B illustrate a body lumen filter 100′ that includes one or more expandable supports 125A, 125B. As illustrated in FIG. 3A, the expanding supports 125A, 125B can include a bistable structure that allows them to move between the stable, pre-deployed state shown in FIG. 3A and the stable, deployed state shown in FIG. 3B. The expandable supports 125A, 125B can be generally annularly shaped members with a sinusoidal pattern imposed thereon to allow for bistable deflection to thereby position a filtering structure 120′ in position to filter a flow of body fluid through a body lumen. In the examples described above bistable anchors have been provided that form a body that moves between bistable states to anchor body lumen filters to a body lumen wall.

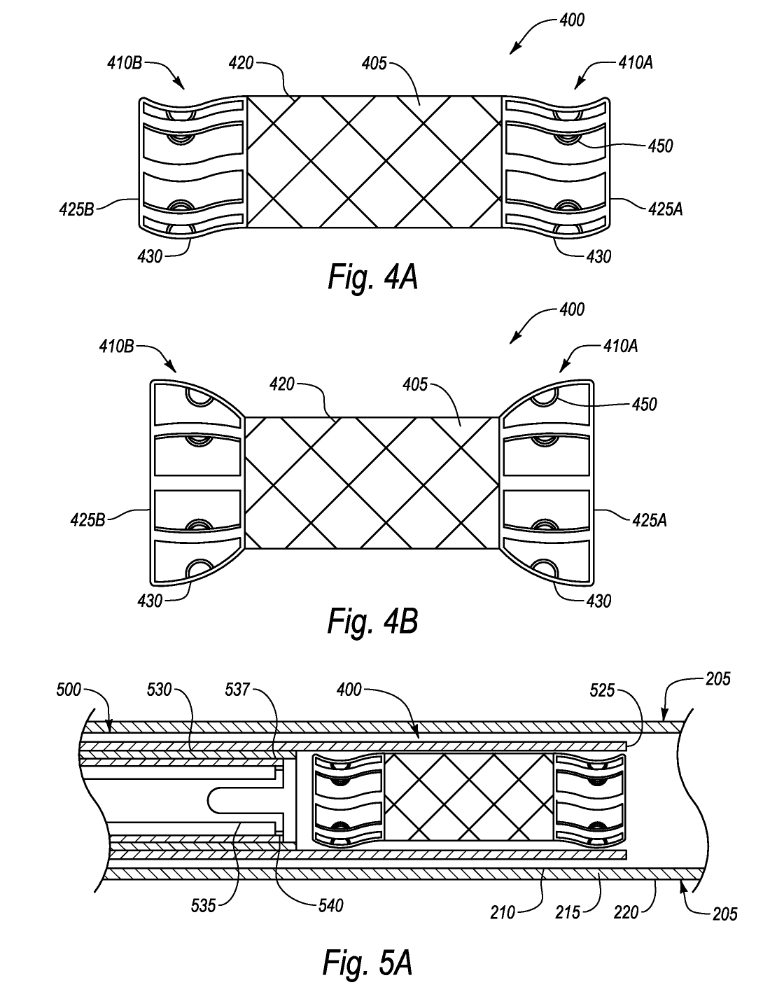

FIGS. 4A-4B illustrate a body lumen filter 400 that includes a body 405 and bistable anchors 410A, 410B operatively associated with the body. In the illustrated example, the body 405 includes a filtering structure formed from a plurality of interlaced and/or interconnected filtering struts 420 that extend at least partially between the bistable anchors 410A, 410B to capture emboli or other particulates.

In at least one example, the bistable anchors 410A, 410B can include annular supports 425A, 425B and bistable struts 430. In at least one example, the bistable struts 430 can be formed by cutting the shape of the bistable struts 430 into the annular supports 425A, 425B. Such a configuration can allow the bistable struts 430 to be deflected from a stable, pre-deployed state shown in FIG. 4A, through a tipping state, and to the stable deployed state shown in FIG. 4B by the application of radial forces, as described above.

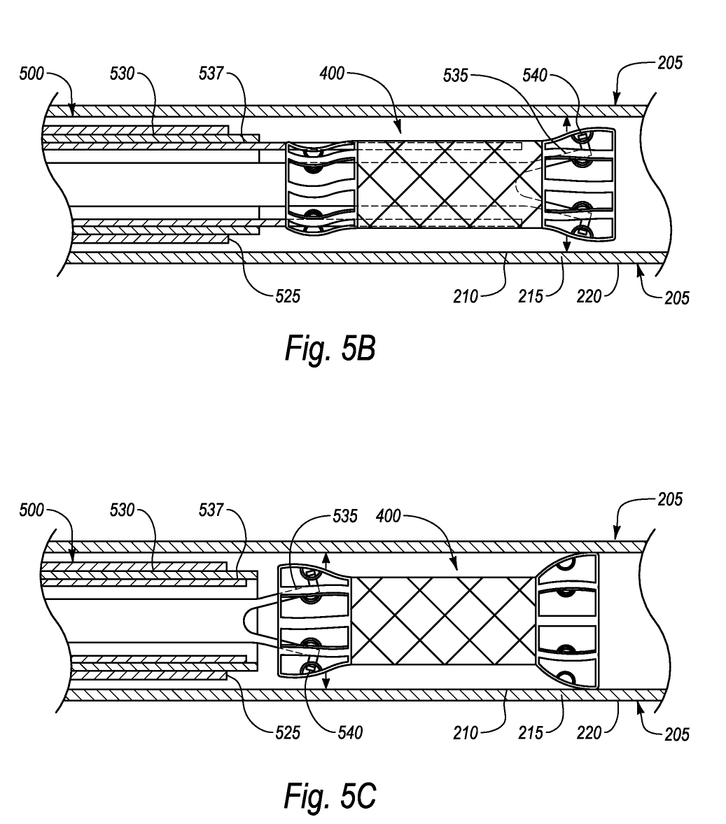

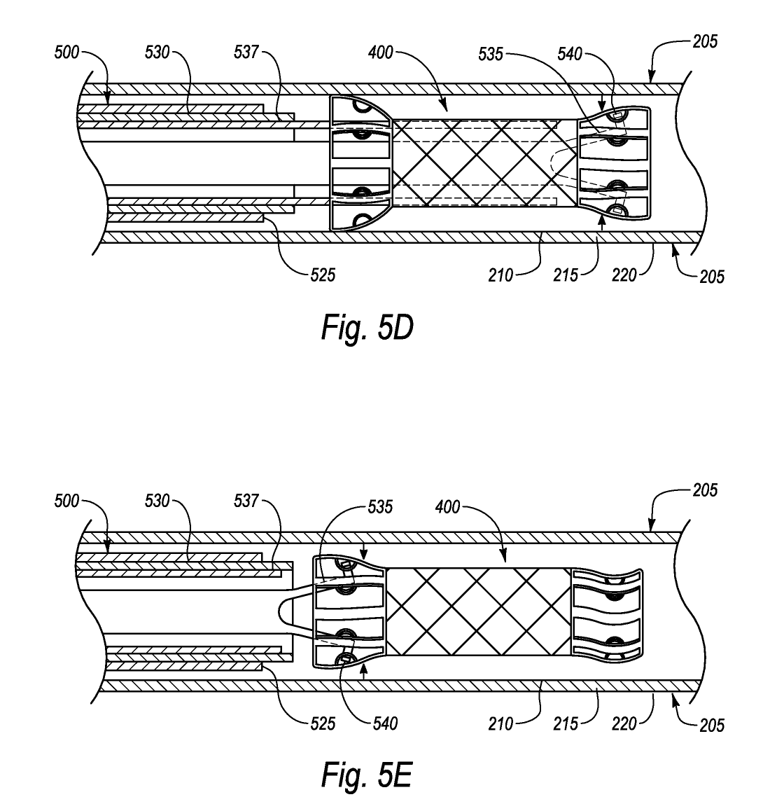

FIGS. 5A-5E illustrate one exemplary method for deploying the body lumen filter 400. FIG. 5A illustrates the body lumen filter 400 in a pre-deployed configuration and located within a deployment device 500. The deployment device 500 is configured to deploy the filter 400 into a body lumen 205. The body lumen 205 may include an inner layer 210 (i.e. intima layer), a medial layer 215, an adventitial layer 220, or combinations thereof. The deployment device 500 can include an outer housing 525 and an inner housing 530 that may be actuated from a proximally located handle (not shown). A radial force mechanism 535 may be positioned within an expansion housing 537 and may similarly be actuated from the proximally located handle. The radial force mechanism 535 can include retrieval features 540 configured to engage a corresponding engagement features 450 (FIG. 4B) on the body lumen filter 400.

As previously discussed, the body lumen filter 400 includes bistable anchors 410A, 410B. To deploy the body lumen filter 400, the deployment device 500 is moved near a desired location within a body lumen 205 by using a catheter or other techniques. In one example, once the deployment device 500 is near the desired location, the inner housing 530 may be advanced distally relative to the outer housing 525, thereby driving the body lumen filter 400 from the outer housing 525 toward the desired location. In another example, the deployment device 500 may be advanced to the desired location, the inner housing 530 may be advanced distally to abut the body lumen filter 400, the outer housing 525 may be retracted, or combinations thereof.

The radial force mechanism 535 can then be positioned relative to one of the bistable anchors 410A, 410B. For ease of reference, the radial force mechanism 535 is shown as being first positioned relative to the distal bistable anchor 410A, though the radial force mechanism 535 could be positioned relative to the proximal bistable anchor 410B.

Once the radial force mechanism 535 is positioned relative to one of the bistable anchors 410A, 410B, the radial force mechanism 535 may be advanced distally relative to the expansion housing 537, the expansion housing 537 may be retracted relative to the radial force mechanism 535, or combinations thereof. In at least one example, the radial force mechanism 535 can be formed of a resilient material shaped such that as the radial force mechanism 535 passes from the expansion housing 537, the radial force mechanism 535 expands as illustrated in FIG. 5B.

As the radial force mechanism 535 expands it comes into contact with the bistable anchor 410A. Continued expansion of the radial force mechanism 535 as it expands can exert sufficient force on the bistable anchor 410A to cause the bistable anchor 410A to deflect past the tipping state described above. Once the radial force mechanism 535 deflects the bistable anchor 410A past the tipping state, the bistable anchors 410 continue to deflect toward the deployed state, illustrated in FIG. 5C.

The radial force mechanism 535 can be collapsed by drawing the radial force mechanism 535 at least partially into the expansion housing 537. As shown in FIG. 5C, radial force mechanism 535 can then be moved into proximity with the other bistable anchor 410B and expanded by being at least partially freed from the expansion housing 537 as described above to expand the bistable anchor 410B to thereby fully deploy the body lumen filter 400. The expansion of the radial force mechanism 535 and other radial force mechanisms illustrated herein can be achieved as the mechanism is freed from the housing 537. The radial force mechanism 535 can be contracted by pulling the mechanism 335 back into the housing 537. This can occur, by way of example only, by advancing/retracting the housing 537 and/or the radial force mechanism 535.

At some point, it may be desirable to retrieve the body lumen filter 400. FIGS. 5D-5E illustrates steps for retrieving the body lumen filter 400. As illustrated in FIG. 5D, retrieving the body lumen filter 400 can include positioning the deployment device 500 such that the expansion housing 537 is positioned in proximity to the bistable anchor 410A and further positioning the radial force mechanism 535 distally of the expansion housing 537. Thereafter, the radial force mechanism 535 can be moved into engagement with the engagement feature 450 on the body lumen filter 400.

In particular, the radial force mechanism 535 can be advanced distally beyond the expansion housing 537 to allow the radial force mechanism 535 to expand. The retrieval features 540 associated with the radial force mechanism 535 can then be moved into engagement with engagement features 450 or some other portion of the bistable anchor 410A. Thereafter, the radial force mechanism 535 can be moved proximally relative to the expansion housing 537 to thereby cause the retrieval features 540 to move radially inward. Engagement between the engagement features 450 and the retrieval feature 540 also causes the bistable anchor 410A to deflect radially inward toward the pre-deployed stable state. Once the bistable anchor 410A transitions to the pre deployed stable state, the radial force mechanism 535 can disengage from the engagement features 450.

Thereafter, as shown in FIG. 5E the retrieval feature 540 can also be moved into engagement with the engagement features 450 on the bistable anchor 410B and collapsed as described above. With the bistable anchors in the pre-deployed stable states, the body lumen filter 400 can be drawn into the outer housing 525 by drawing the radial force mechanism 535 within the outer housing 525. Once the body lumen filter 200 is located within the deployment device 500, the deployment device 500 can be removed to thereby complete retrieval of the body lumen filter 400. Accordingly, various configurations of body lumen filters 400 can include bistable anchors.

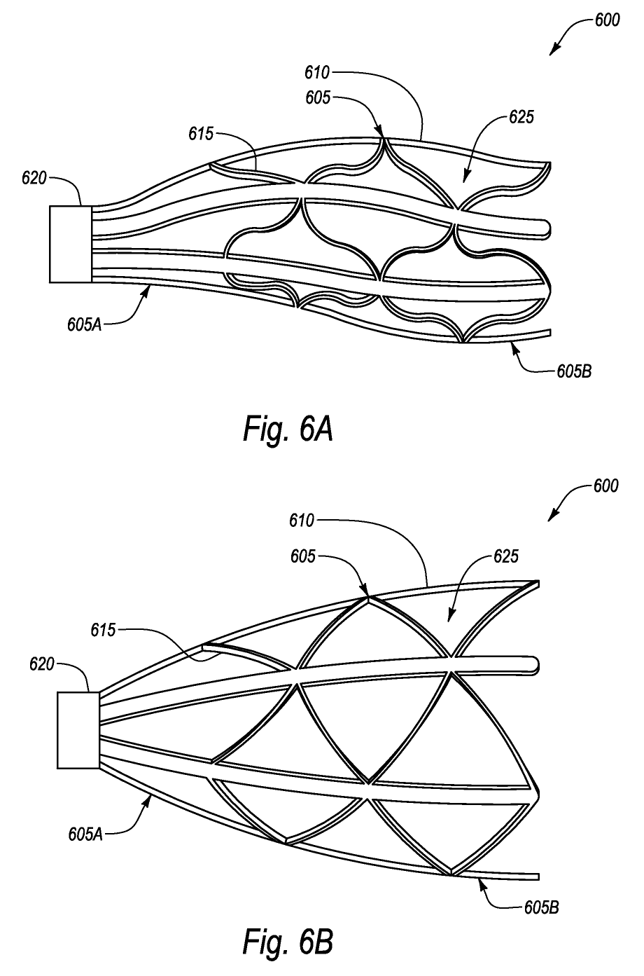

FIG. 6A illustrates another example of a body lumen filter 600 with a body 605 formed from bistable anchors 610. Bistable anchors 610 that extend between a distal or first end 605A and a proximal or second end 605B. Lateral struts 615 can be operatively associated with the bistable anchors 610 to couple adjacent bistable anchors 610. The bistable anchors 610 can be coupled together near the first end 605A by a ring 620. The bistable anchors 610 can be configured to deflect from the stable, pre-deployed state show in FIG. 6A to the stable, deployed state shown in FIG. 6B in a similar manner as described above.

As the bistable anchors 610 move to the deflected state, the ring 620 can help maintain the bistable anchors 610 relatively close together near the first end 605A. As a result, as the bistable anchors 610 extend away from the ring 620 toward the second end 605B, the bistable anchors 610 begin to separate in a radial direction. The lateral struts 615 and the bistable anchors 610 are coupled so as to form filter openings 625 while the body lumen filter 600 is deployed, as shown in FIG. 6B.

The lateral struts 615 can be configured as active bistable structures, passive structures, or combinations thereof. In active or combination structures, the lateral struts 615 can provide supplemental deflection to the bistable anchors 610 as the body lumen filter 600 moves between the states shown in FIGS. 6A and 6B. In other examples, the lateral struts 615 can provide the deflection associated with moving the body lumen between those states. The body lumen filter 600 can be moved between stable states as desired, including through the use of deployment devices as described above. Accordingly, the body lumen filter 600 can be deployed as desired within a body lumen.

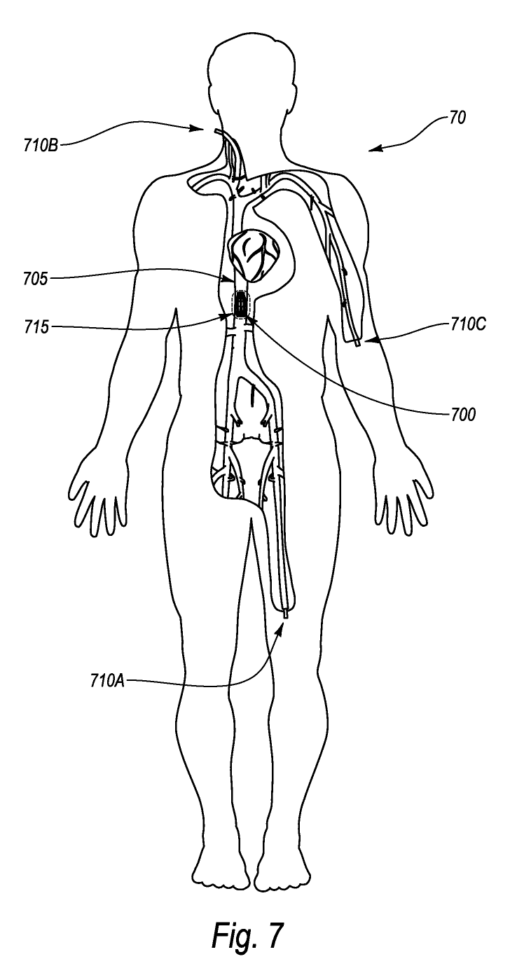

FIG. 7 illustrates an exemplary subject 70 for an implantable lumen filter 700. The implantable lumen filter 700 may be functionally similar to the implantable lumen filters previously described above, wherein certain features will not be described in relation to this configuration wherein those components may function in the manner as described above and are hereby incorporated into the configuration described below. Like structures and/or components are given like reference numerals. Additionally, the implantable lumen filter 700 may incorporate at least one bistable component of the implantable lumen filters described above.

Although many of the embodiments herein may describe an implantable lumen filter 700, other filters may be deployed and/or retrieved using at least one embodiment of a filter retrieval system described herein. The filter 700 may be implanted in a body lumen 705 of the subject 70. The filter 700 may be inserted and/or retrieved through an access site 710A, 710B, 710C. In the present embodiment, the access site may include a femoral artery access site 710A, a jugular vein access site 710B, a radial vein access site 710C, femoral vein, brachial vein, brachial artery, other access sites, or combinations thereof. For instance, the filter 700 may be inserted through the femoral artery access site 710A and retrieved through the jugular or radial vein access site 710B, 710C. In another example, the filter 700 may be inserted through the jugular vein access site 710B and retrieved through the femoral artery or radial vein access site 710A, 710C. In a further example, the filter 700 may be inserted through the radial vein access site 710C and retrieved through the femoral artery or jugular vein access site 710A, 710B.

The filter 700 may be inserted and retrieved through the radial vein access site 710C. Additionally, the filter 700 may be inserted and retrieved through the jugular vein access site 710B. Further, the filter 700 may be inserted and retrieved through the femoral artery access site 710A.

The filter 700 may be deployed near a deployment site 715. In the present embodiment, the deployment site 715 may include a location within the inferior vena cava. In other embodiments, other deployment sites may be used, such as the superior vena cava. For example, the deployment site 715 may include all larger veins.

As mentioned above, some body lumen filters typically use jugular, antecubital, or other access sites for retrieval because they are typically not configured to be retrieved through the femoral access. Retrieval through the same access site through which the filter was deployed may be desired. At least one embodiment of a filter retrieval system may provide for retrieval through the same access site through which the filter was deployed.

Embodiments of the filters and/or anchors disclosed herein can include or be manufactured from a material made from any of a variety of known suitable materials such as copper-zinc-aluminum; copper-aluminum-nickel; nickel-titanium (NiTi) alloys known as nitinol; nickel-titanium platinum; nickel-titanium palladium; and cobalt-chromium-nickel alloys or cobalt-chromium-nickel-molybdenum alloys known as elgiloy alloys. For example, a body lumen filter and/or anchor may be, at least partially, formed from various materials including, but not limited to, stainless steel, cobalt chromium and/or alloys thereof, niobium tantalum and/or alloys thereof, other materials suitable for implantable stents, filters, or other implantable medical devices, and/or combinations thereof suitable for the forming bistable anchors. In addition, a body lumen filter and/or anchor may be, at least partially, formed of or include a radiopaque material and/or be coated with a radiopaque material to enhance visibility of the body lumen filter and/or the anchors.

Also, the filters can be comprised of a variety of known suitable deformable materials, including stainless steel, silver, platinum, tantalum, palladium, nickel, titanium, nitinol, nitinol having tertiary materials, niobium-tantalum alloy optionally doped with a tertiary material, cobalt-chromium alloys, or other known biocompatible materials. Such biocompatible materials can include a suitable biocompatible polymer in addition to or in place of a suitable metal. A device or member can include biodegradable or bioabsorbable materials, which can be either plastically deformable or capable of being set in the deployed configuration. If plastically deformable, the material can be selected to allow the device or member to be expanded in a similar manner using an expandable member so as to have sufficient radial strength and also to reduce recoil once expanded. If the polymer is to be set in the deployed configuration, the expandable member can be provided with a heat source or infusion ports to provide the required catalyst to set or cure the polymer.

In one embodiment, the filters, including the expander/removal device and/or the expansion members, can be made from a superelastic alloy such as nickel-titanium or nitinol, and may include a ternary element selected from the group of chemical elements consisting of iridium, platinum, gold, rhenium, tungsten, palladium, rhodium, tantalum, silver, ruthenium, or hafnium. The added ternary element improves the radiopacity of the nitinol closure device or other medical device, including the expander/removal device and/or the expansion members, comparable to that of a stainless steel device or member of the same size and shape coated with a thin layer of gold. The nitinol device or member may have improved radiopacity yet may retain its superelastic and shape memory behavior and further maintains a thin strut/wall thickness for high flexibility.

Furthermore, the closure device body or other medical device, including the expander/removal device and/or the expansion members, can be formed from a ceramic material. In one aspect, the ceramic can be a biocompatible ceramic that optionally can be porous. Examples of suitable ceramic materials include hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like. Optionally, the ceramic can be provided as sinterable particles that are sintered into the shape of a closure device or layer thereof.

These materials may include at least one beneficial agent incorporated into the material and/or coated over at least a portion of the material. The beneficial agents may be applied to body lumen filters that have been coated with a polymeric compound. Incorporation of the compound or drug into the polymeric coating of the body lumen filter can be carried out by dipping the polymer-coated body lumen filter into a solution containing the compound or drug for a sufficient period of time (such as, for example, five minutes) and then drying the coated body lumen filter, preferably by means of air drying for a sufficient period of time (such as, for example, 30 minutes). The polymer-coated body lumen filter containing the beneficial agent may then be delivered to a body vessel.

The pharmacologic agents that can be effective in preventing restenosis can be classified into the categories of anti-proliferative agents, anti-platelet agents, anti-inflammatory agents, anti-thrombotic agents, and thrombolytic agents. Anti-proliferative agents may include, for example, crystalline rapamycin. These classes can be further sub-divided. For example, anti-proliferative agents can be anti-mitotic. Anti-mitotic agents inhibit or affect cell division, whereby processes normally involved in cell division do not take place. One sub-class of anti-mitotic agents includes vinca alkaloids. Representative examples of vinca alkaloids include, but are not limited to, vincristine, paclitaxel, etoposide, nocodazole, indirubin, and anthracycline derivatives, such as, for example, daunorubicin, daunomycin, and plicamycin. Other sub-classes of anti-mitotic agents include anti-mitotic alkylating agents, such as, for example, tauromustine, bofumustine, and fotemustine, and anti-mitotic metabolites, such as, for example, methotrexate, fluorouracil, 5-bromodeoxyuridine, 6-azacytidine, and cytarabine. Anti-mitotic alkylating agents affect cell division by covalently modifying DNA, RNA, or proteins, thereby inhibiting DNA replication, RNA transcription, RNA translation, protein synthesis, or combinations of the foregoing.

Anti-platelet agents are therapeutic entities that act by (1) inhibiting adhesion of platelets to a surface, typically a thrombogenic surface, (2) inhibiting aggregation of platelets, (3) inhibiting activation of platelets, or (4) combinations of the foregoing. Activation of platelets is a process whereby platelets are converted from a quiescent, resting state to one in which platelets undergo a number of morphologic changes induced by contact with a thrombogenic surface. These changes include changes in the shape of the platelets, accompanied by the formation of pseudopods, binding to membrane receptors, and secretion of small molecules and proteins, such as, for example, ADP and platelet factor 4. Anti-platelet agents that act as inhibitors of adhesion of platelets include, but are not limited to, eptifibatide, tirofiban, RGD (Arg-Gly-Asp)-based peptides that inhibit binding to gpIIbIIIa or αvβ3, antibodies that block binding to gpIIaIIIb or αvβ3, anti-P-selectin antibodies, anti-E-selectin antibodies, compounds that block P-selectin or E-selectin binding to their respective ligands, saratin, and anti-von Willebrand factor antibodies. Agents that inhibit ADP-mediated platelet aggregation include, but are not limited to, disagregin and cilostazol.

Anti-inflammatory agents can also be used. Examples of these include, but are not limited to, prednisone, dexamethasone, hydrocortisone, estradiol, fluticasone, clobetasol, and non-steroidal anti-inflammatories, such as, for example, acetaminophen, ibuprofen, naproxen, and sulindac. Other examples of these agents include those that inhibit binding of cytokines or chemokines to the cognate receptors to inhibit pro-inflammatory signals transduced by the cytokines or the chemokines Representative examples of these agents include, but are not limited to, anti-IL1, anti-IL2, anti-IL3, anti-IL 4, anti-IL8, anti-IL15, anti-IL18, anti-GM-CSF, and anti-TNF antibodies.

Anti-thrombotic agents include chemical and biological entities that can intervene at any stage in the coagulation pathway. Examples of specific entities include, but are not limited to, small molecules that inhibit the activity of factor Xa. In addition, heparinoid-type agents that can inhibit both FXa and thrombin, either directly or indirectly, such as, for example, heparin, heparin sulfate, low molecular weight heparins, such as, for example, the compound having the trademark Clivarin®, and synthetic oligosaccharides, such as, for example, the compound having the trademark Arixtra®. Also included are direct thrombin inhibitors, such as, for example, melagatran, ximelagatran, argatroban, inogatran, and peptidomimetics of binding site of the Phe-Pro-Arg fibrinogen substrate for thrombin. Another class of anti-thrombotic agents that can be delivered is factor VII/VIIa inhibitors, such as, for example, anti-factor VII/VIIa antibodies, rNAPc2, and tissue factor pathway inhibitor (TFPI).

Thrombolytic agents, which may be defined as agents that help degrade thrombi (clots), can also be used as adjunctive agents, because the action of lysing a clot helps to disperse platelets trapped within the fibrin matrix of a thrombus. Representative examples of thrombolytic agents include, but are not limited to, urokinase or recombinant urokinase, pro-urokinase or recombinant pro-urokinase, tissue plasminogen activator or its recombinant form, and streptokinase.

One or more immunosuppressant agents may be used. Immunosuppressant agents may include, but are not limited to, IMURAN® azathioprine sodium, brequinar sodium, SPANIDIN® gusperimus trihydrochloride (also known as deoxyspergualin), mizoribine (also known as bredinin), CELLCEPT® mycophenolate mofetil, NEORAL® Cylosporin A (also marketed as different formulation of Cyclosporin A under the trademark SANDIMMUNE®), PROGRAF® tacrolimus (also known as FK-506), sirolimus and RAPAMUNE®, leflunomide (also known as HWA-486), glucocorticoids, such as prednisolone and its derivatives, antibody therapies such as orthoclone (OKT3) and Zenapax®, and antithymyocyte globulins, such as thymoglobulins. In addition, a crystalline rapamycin analog, A-94507, SDZ RAD (a.k.a. Everolimus), and/or other immunosuppressants.

The present invention can be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A body lumen filter, comprising:
a plurality of bistable anchors configured to move between a stable pre-deployed state and a stable deployed state wherein in the stable deployed state the stable pre-deployed diameter the bistable anchors define a pre-deployed anchor diameter and in the stable deployed diameter the bistable anchors define a deployed anchor diameter, the deployed diameter being larger than the pre-deployed diameter; and
a filtering structure operatively associated with the bistable anchors.

2. The body lumen filter of claim 1, wherein the bistable anchors have a generally sinusoidal shape in the stable pre-deployed state and a generally arcuate shape in the stable deployed state.

3. The body lumen filter of claim 1, further comprising lateral struts coupling at least one pair of adjacent bistable anchors.

4. The body lumen filter of claim 3, wherein at least one of the lateral struts has a bistable configuration.

5. The body lumen filter of claim 4, wherein the lateral struts and the bistable anchors form a plurality of filtering openings defining at least a portion of the filtering structure.

6. The body lumen filter of claim 1, wherein the bistable anchors form a body of the body lumen filter.

7. The body lumen filter of claim 6, further comprising a support coupling the filtering structure to the bistable anchors.

8. The body lumen filter of claim 7, wherein the support comprises a bistable expandable annular support.

9. The body lumen filter of claim 6, wherein the body includes a first end and a second end wherein in the stable deployed state the first end is narrower than the second end.

10. The body lumen filter of claim 1, wherein the filtering structure forms a body having a first end and a second end and wherein at least one bistable anchor is operatively associated with at least one of the first end and the second end.

11. The body lumen filter of claim 10, wherein the filtering structure includes a plurality of struts.

12. The body lumen filter of claim 10, wherein the bistable anchor includes axially opposing annular supports and bistable struts positioned between the annular supports.

13. A system, comprising:
a body lumen filter having a plurality of bistable anchors and a filtering structure operatively associated with the bistable anchors; and
a deployment device configured to move the bistable anchors between a stable pre-deployed state past, a transition state, and a stable deployed state wherein in the stable deployed state the stable pre-deployed diameter the bistable anchors define a pre-deployed anchor diameter and in the stable deployed diameter the bistable anchors define a deployed anchor diameter, the deployed diameter being larger than the pre-deployed diameter.

14. The system of claim 13, wherein the bistable anchors form a body of the body lumen filter.

15. The system of claim 14, further comprising a ring-shaped support coupling the body and the filtering structure.

16. The system of claim 13, wherein the bistable anchors are positioned on opposing ends of the body lumen filter.

17. The system of claim 13, wherein in the stable deployed state one end of the body lumen filter is narrower than an opposing end of the body lumen filter.

18. A method for filtering a body lumen, the method comprising:
providing a body lumen filter, comprising:
a plurality of bistable anchors configured to move between a stable pre-deployed state and a stable deployed state wherein in the stable deployed state the stable pre-deployed diameter the bistable anchors define a pre-deployed anchor diameter and in the stable deployed diameter the bistable anchors define a deployed anchor diameter, the deployed diameter being larger than the pre-deployed diameter, and a filtering structure operatively associated with the bistable anchors;
moving the bistable anchors to the pre-deployed state and positioning the body lumen filter within a deployment device;
delivering the body lumen filter to a desired deployment site within the body lumen; and
moving the bistable anchors from the pre-deployed state through a transition state to cause the bistable anchors to move toward the deployed state to cause the bistable anchors to exert radial forces to an inner wall of the body lumen.

19. The method of claim 18, further comprising applying a force to move the bistable anchors through the transition state to the stable pre-deployed state, positioning the body lumen filter within a deployment device, and removing the body lumen filter from the desired deployment site within the body lumen.

20. The method of claim 18, wherein moving the bistable anchor from the pre-deployed state through a transition state include applying a radially outward force.
